(19) United States
(12) Patent Application Publication
(10) Pub. No.: US 2018/0030206 A1
(43) Pub. Date: Feb. 1, 2018

(54) THERMOPLASTIC COPOLYIMIDES

(71) Applicant: RHODIA OPERATIONS, Paris (FR)

(72) Inventor: Stéphane JEOL, Cumming, GA (US)

(21) Appl. No.: 15/730,414

(22) Filed: Oct. 11, 2017

Related U.S. Application Data
(63) Continuation of application No. 14/345,070, filed on Sep. 25, 2014.

(30) Foreign Application Priority Data
Sep. 20, 2011 (FR) 1158326

(51) Int. Cl. C08G 73/10; C07C 211/09; C07C 63/313; B29C 49/04; C08K 3/00; B29C 45/00; B29K 105/00; B29K 79/00

(52) U.S. Cl. CPC C08G 73/1042; C08K 3/0033; C08G 73/1082; C08G 73/1075; C07C 211/09; B29C 45/0001; C07C 63/313; B29C 49/04; B29K 2079/08; B29K 2105/0085

(57) ABSTRACT

The present invention relates to semiaromatic semicrystalline thermoplastic copolyimides obtained by polymerization of at least: (a) an aromatic compound comprising two anhydride functions and/or carboxylic acid and/or ester derivatives thereof; (b) a diamine of formula (I) NH2—R—NH2 in which R is a divalent aliphatic hydrocarbon-based radical optionally comprising heteroatoms, the two amine functions being separated by a number X of carbon atoms, X being between 4 and 12; and (c) a diamine of formula (II) NH2—R′—NH2 in which R′ is a divalent aliphatic hydrocarbon-based radical optionally comprising heteroatoms, the two amine functions being separated by a number Y of carbon atoms, Y being between 10 and 20; it being understood that diamine (b) is different from diamine (c).

The present invention relates to semiaromatic semicrystalline thermoplastic copolyimides obtained by polymerization of at least: (a) an aromatic compound comprising two anhydride functions and/or carboxylic acid and/or ester derivatives thereof; (b) a diamine of formula (I) NH2—R—NH2 in which R is a divalent aliphatic hydrocarbon-based radical optionally comprising heteroatoms, the two amine functions being separated by a number X of carbon atoms, X being between 4 and 12; and (c) a diamine of formula (II) NH2—R′—NH2 in which R′ is a divalent aliphatic hydrocarbon-based radical optionally comprising heteroatoms, the two amine functions being separated by a number Y of carbon atoms, Y being between 10 and 20; it being understood that diamine (b) is different from diamine.

### PRIOR ART

Technical polyamides are used for the preparation of numerous articles in various fields, such as the motor vehicle field, where specific properties of stiffness, impact strength, size stability, in particular at relatively high temperature, surface appearance, density and weight are particularly desired. The choice of a material for a given application is generally guided by the level of performance required with regard to certain properties and by its cost. Specifically, there is an ongoing search for novel materials that are capable of meeting specifications in terms of performance and/or costs.

However, certain polyamides have strong water uptake, which gives rise to problems linked to the size stability of the articles used in many applications. Certain polyamides also have insufficient heat resistance, especially a thermomechanical strength preventing their use in applications in which there are constraints of this type to be respected.

There is thus a need to overcome these drawbacks while at the same time using polymers whose melting points are compatible with the transformation temperatures of standard thermoplastic polyamides, a melting point generally below 330° C., and which can thus be transformed via the implementation processes known for thermoplastics, similar to polyamides, while at the same time benefiting from excellent heat resistance.

Certain polyimides were known in the prior art to attempt to solve this problem, but had implementation temperatures that were too high for them to be transformed via polyamide implementation processes. Moreover, the use of such temperatures leads to significant degradation of the polyimide matrix and to colorations that are detrimental for producing esthetic components. What is more, their high melting points prevent the use of certain additives, for instance organophosphorus fire retardants or natural fibers which decompose at such temperatures.

### INVENTION

It has just been demonstrated by the Applicant that it is possible to prepare particular semiaromatic, semicrystalline thermoplastic copolyimides by using, as constituent monomers, at least two types of diamines bearing in their main chain from 4 to 12 carbon atoms, and from 10 to 20 carbon atoms, respectively.

These copolyimides have melting points that are entirely compatible with the transformation temperatures of standard thermoplastic polyamides, the copolyimides according to the invention preferentially having a melting point Tf of between 50 and 330° C. These copolyimides moreover have high crystallization temperatures enabling the production cycle times to be significantly reduced.

The copolyimides according to the invention preferentially have a glass transition temperature Tg of between -50° C. and +170° C.

These copolyimides obtained are semicrystalline and thermoplastic and have the property of not releasing or absorbing water during the subsequent transformation steps, for instance pultrusion, extrusion or injection-molding. These copolyimides are particularly hydrophobic and thus have excellent size stability.

The present invention thus relates to a semiaromatic semicrystalline thermoplastic copolyimide obtained by polymerization of at least:
(a) an aromatic compound comprising two anhydride functions and/or carboxylic acid and/or ester derivatives thereof; and
(b) a diamine of formula (I) NH2—R—NH2 in which R is a saturated or unsaturated divalent aliphatic hydrocarbon-based radical, optionally comprising heteroatoms, the two amine functions being separated by a number X of carbon atoms, X being between 4 and 12 (limits included); and
(c) a diamine of formula (II) NH2—R′—NH2 in which R′ is a saturated or unsaturated divalent aliphatic hydrocarbon-based radical, optionally comprising heteroatoms, the two amine functions being separated by a number Y of carbon atoms, Y being between 10 and 20 (limits included), the radical R′ comprising not more than 20 carbon atoms;
   it being understood that diamine (b) is different from diamine (c).

According to a first embodiment, the invention relates to a semiaromatic, semicrystalline thermoplastic copolyimide obtained by polymerization of at least two ammonium carboxylate salts obtained from monomers (a), on the one hand, and (b) and (c), on the other hand, in which (a) is an aromatic compound comprising 4 carboxylic acid functions; (b) is a diamine of formula (I) NH2—R—NH2 in which R is a saturated or unsaturated divalent aliphatic hydrocarbon-based radical, optionally comprising heteroatoms, the two amine functions being separated by a number X of carbon atoms, X being between 4 and 12 (limits included); and (c) is a diamine of formula (II) NH2—R′—NH2 in which R′ is a saturated and/or unsaturated divalent aliphatic hydrocarbon-based radical, optionally comprising heteroatoms, the two amine functions being separated by a number Y of carbon atoms, Y being between 10 and 20 (limits included), the radical R′ comprising not more than 20 carbon atoms; it being understood that diamine (b) is different from diamine (c). Most particularly, the polymerization involves two ammonium carboxylate salts, which are optionally imbalanced and/or bear a chain limiter.

The invention also relates to a process for manufacturing a semiaromatic semicrystalline thermoplastic copolyimide obtained by polymerization of at least the monomers mentioned previously, in particular in the form of salts, and more particularly by solid-state polymerization.

The invention also relates to copolyimides that may be obtained via the process as described previously.

### Definitions

The term “semicrystalline” refers to a copolyimide having an amorphous phase and a crystalline phase, for example having a degree of crystallinity of between 1% and 85%.

The term “thermoplastic copolyimide” means a copolyimide having a temperature above which the material softens and melts, and below which it becomes hard.

The determination of the melting point of the copolyimide is preferably performed by measuring the temperature at the peak of the melting endotherm measured by differential scanning calorimetry (DSC), using a Perkin-Elmer Pyris 1 machine, heating the copolyimide from 20° C. at a rate of 10° C./minute.

The copolyimides obtained from only one diamine and from an aromatic compound comprising two anhydride functions or derivatives are polyimides, generally known as homopolyimides. The reaction between at least three different monomers produces a copolyimide. Copolyimides may be defined by the molar composition of each constituent monomer.

### Monomers

Compounds (a) preferentially bear carboxylic acid functions in positions such that they can generally form two acid anhydride functions on the same molecule via a dehydration reaction. The compounds of the present invention generally bear two pairs of carboxylic acid functions, each pair of functions being linked to an adjacent carbon atom, α and β. Tetracarboxylic acid functions may be obtained from dianhydrides by hydrolysis of the acid anhydride functions. Examples of acid dianhydrides and of tetracarboxylic acids, derived from dianhydrides, are described in patent U.S. Pat. No. 7,932,012.

Compounds (a) of the invention may also bear functional groups, especially, for instance, the group —SO3X, with X═H or a cation, such as Na, Li, Zn, Ag, Ca, Al, K or Mg.

The aromatic compounds comprising two anhydride functions are preferentially chosen from the group consisting of: pyromellitic anhydride, 3,3′,4,4′-biphenyltetracarboxylic dianhydride, 2,3,3′,4′-biphenyltetracarboxylic dianhydride, 2,2′,3,3′-biphenyltetracarboxylic dianhydride, 3,3′,4,4′-benzophenonetetracarboxylic dianhydride, 2,2′,3,3′-benzophenonetetracarboxylic dianhydride, 1,2,5,6-naphthalenetetracarboxylic dianhydride, 2,3,6,7-naphthalenetetracarboxylic dianhydride and 2,2′-bis(3,4-dicarboxyphenyl)hexafluoropropanetetracarboxylic dianhydride.

The aromatic compounds comprising carboxylic acid functions derived from two anhydride functions are preferably chosen from the group consisting of: pyromellitic acid, 3,3′,4,4′-biphenyltetracarboxylic acid, 2,3,3′,4′-biphenyltetracarboxylic acid, 2,2′,3,3′-biphenyltetracarboxylic acid, 3,3′,4,4′-benzophenonetetracarboxylic acid, 2,2′,3,3′-benzophenonetetracarboxylic acid, 1,2,5,6-naphthalenetetracarboxylic acid, 2,3,6,7-naphthalenetetracarboxylic acid, 2,3,5,6-pyridinetetracarboxylic acid, 3,4,9,10-perylenetetracarboxylic acid, 3,3′,4,4′-tetraphenylsilanetetracarboxylic acid, 2,2′-bis(3,4-bicarboxyphenyl)hexafluoropropanetetracarboxylic acid.

Compounds (a) of the invention may also be free of functional groups other than carboxylic acids. Advantageously, compounds (a) are tetracarboxylic acids whose carboxylic functions are such that they can give rise to two anhydride functions via a dehydration reaction.

Compounds (a) may comprise only one aromatic ring.

Diamines (b) and (c) are aliphatic diamines. For the purposes of the present invention, the term “aliphatic diamine” means a compound in which the amine functions are each borne by an aliphatic carbon, in particular by an sp3 carbon. Most particularly, the amine functions are primary amines. Most particularly, the aliphatic diamines comprise a saturated aliphatic group R.

Diamines (b) and (c) of the present invention thus bear a main chain separating the two amine functions and optionally one or more pendent chains or “side chains”. In the case of diamine (b), the main chain comprises between 4 and 12 carbon atoms. In the case of diamine (c), the main chain comprises between 10 and 20 carbon atoms.

The radicals R and R′, independently of each other, may be saturated or unsaturated, linear or branched, and optionally comprising heteroatoms. The radicals R and R′, independently of each other, may optionally contain one or more heteroatoms, such as O, N, P or S, and/or one or more functional groups such as hydroxyl, sulfone, ketone, ether or other functions.

Diamines (b) of the invention preferentially bear two primary amine functions. Diamines (c) of the invention preferentially bear two primary amine functions.

Diamine (b) is preferentially chosen from the group consisting of: 1,4-diaminobutane, 1,5-diaminopentane, 2-methyl-1,5-diaminopentane, hexamethylenediamine, 3-methylhexamethylenediamine, 2,5-dimethylhexamethylenediamine, 2,2,4- and 2,4,4-trimethyl-hexamethylenediamine, 1,7-diaminoheptane, 1,8-diaminooctane, 2,2,7,7-tetramethyloctamethylenediamine, 1,9-diaminononane, 5-methyl-1,9-diaminononane, 1,10-diaminodecane, 1,11-diaminoundecane, 1,12-diaminododecane and 5-methyl-1,9-diaminononane.

Diamine (c) is preferentially chosen from the group consisting of: 1,10-diaminodecane, 1,11-diaminoundecane, 1,12-diaminododecane, 1,13-diaminotridecane, 1,14-diaminotetradecane, 1,15-diaminopentadecane, 1,16-diaminohexadecane, 1,17-diaminoheptadecane, 1,18-diaminooctodecane, 1,19-diaminononadecane and 1,20-diaminoeicosane. Advantageously, diamine (c) is chosen from 1,13-diaminotridecane, 1,14-diaminotetradecane, 1,15-diaminopentadecane, 1,16-diaminohexadecane, 1,17-diaminoheptadecane, 1,18-diaminooctodecane, 1,19-diaminononadecane and 1,20-diaminoeicosane, and even more particularly from 1,14-diaminotetradecane, 1,15-diaminopentadecane, 1,16-diaminohexadecane, 1,17-diaminoheptadecane, 1,18-diaminooctodecane, 1,19-diaminononadecane and 1,20-diaminoeicosane.

Examples of diamines containing heteroatoms that may be mentioned include polyetherdiamines such as Jeffamine® and Elastamine® sold by Huntsman. A variety of polyethers exist, composed of ethylene oxide, propylene oxide or tetramethylene oxide units.

It is possible to obtain copolyimides by using different types of monomers (a), (b) and/or (c); or even to add other types of monomers that are suitable also for obtaining imide functions.

The monomers (a), (b) and/or (c) may be in salified or non-salified form. It is entirely possible to prepare one or more ammonium carboxylate salts formed by reaction between the monomers (a), (b) and/or (c) mentioned previously. Mention may be made, for example, of a mixture comprising monomer (a), monomer (c) and a salt formed by reaction between monomers (a) and (b); or alternatively a mixture comprising monomer (a), monomer (b) and a salt formed by reaction between monomers (a) and (c). Mention may also be made of a mixture between a salt formed by reaction between monomers (a) and (b) and a salt formed by reaction between monomers (a) and (c).

For the purposes of the present invention, the term “ammonium carboxylate salt” means a salt in which the diamine and tetraacid species are linked solely via polar interactions, in particular of the type —COO— H3+N−, and not via covalent bonding. More particularly, the salt comprises a tetraacid and a diamine, which are not linked via covalent bonding. In particular, the salt may have the following structure, with Ar representing an aromatic group:

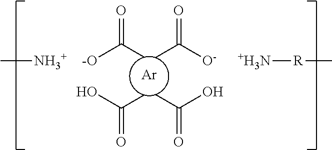

Such a salt may be synthesized in various ways, known to those skilled in the art.

It is possible, for example, to perform an addition of the diamines either simultaneously or one after the other, or sequentially in a solution comprising compound (a). It is also possible to dissolve compound (a) in a solvent such as an alcohol, for instance ethanol or methanol, and to do the same for the diamines. These two solutions are then mixed together with stirring. The ammonium carboxylate salt formed may be insoluble in the solvent used and thus precipitate out. The salt may then be recovered by filtration, washed and dried, and optionally ground.

It is also possible to make a solution of the ammonium carboxylate salt and then to concentrate it while hot and then cool it. The salt then crystallizes and the crystals are recovered and dried. Concentration of the solution may be obtained by evaporating off the solvent such as water or the alcohol or, according to another process, by adding compound (a) and/or the diamines. It is also possible to perform saturation of the solution, i.e. to perform a process for modifying the concentration of the salt in the solution to a value that is compatible with its crystallization. Generally, this concentration is at least equal to and more preferentially greater than the saturation concentration of the salt at the temperature under consideration. More precisely, this concentration corresponds to supersaturation of the salt solution. It is also possible to work at a pressure that enables the solvent of the solution, such as water or the alcohol, to evaporate off, so as to saturate the solution and bring about crystallization. It is also possible to saturate the solution by successive or simultaneous addition of a stream of compound (a) and of a stream of diamines to a salt solution.

By way of example, compound (a) is dissolved in the alcohol, for instance ethanol, in a first medium. Diamine (b) and diamine (c) are dissolved in alcohol in another medium and the two media are then mixed together with stirring. The salt obtained precipitates out.

At the end of this synthesis, the salt may be in the form of a dry powder, in the form of a powder dispersed in a solvent, or dissolved in solution. The salt may be recovered by filtration in the case of a precipitate, and the filter cake may be disintegrated, if necessary. When the salt is dissolved in solution, it may be recovered via a crystallization process by concentration or supersaturation or by making it precipitate out by addition of a non-solvent. The crystalline salt may then be recovered by filtration and the filter cake may be disintegrated, if necessary. Another process for recovering the dispersed particles of dry salt is spraying of the solution, i.e. in particular an operation of sudden evaporation of the solvent sprayed in the form of fine droplets so as to recover the dispersed salt particles.

As regards the mixing of the three different comonomers, it is possible, for example, to perform mixing of preformed salts by preparing different salts of diamines and of compound (a), and thus mixing the salts in water and/or the alcohol. The mixing of the salts may take place homogeneously or heterogeneously.

To do this, it is also possible to place in contact the individual monomers, at different moments of introduction; for example, all at the same time or one after the other or in a well-defined introduction sequence. Thus, it is possible, for example, to introduce a mixture of the two diamines into a solution comprising compound (a). It is also possible first to introduce a first diamine into a solution comprising compound (a), and then to introduce the second diamine. It is also possible to introduce a portion of the first diamine into a solution comprising compound (a), and then a portion of the second diamine, and then another portion of the first diamine, and finally the last portion of the second diamine.

To do this, it is also possible to place in contact one of the comonomers (a), (b) or (c) with a preformed salt of the other two comonomers.

Finally, it is possible to screen the salt particle size, for example by sifting or milling.

The polymerization process may be performed according to the standard processes known to those skilled in the art.

According to an advantageous variant, it is possible to perform a polymerization of the salts in the solid state. The fundamental principle of these processes consists in bringing the starting salt, in air or under an inert atmosphere or under vacuum, to a temperature below its melting point but sufficient to allow the polymerization reaction, generally above the glass transition temperature of the copolyimide. Such a polymerization process may thus comprise, in brief:
a) heating of the product by conductive or convective diffusion or by radiation,
b) inertizing by applying a vacuum, flushing with a neutral gas such as nitrogen, CO2 or superheated steam, or application of a positive pressure,
c) removing the condensation by-product by evaporation, and then flushing with the carrier gas or concentrating the gas phase,
d) stirring mechanically or fluidizing the solid phase with the carrier gas or vibration may be desirable in order to improve the heat and mass transfers and also to prevent any risk of aggregation of the divided solid.

According to a particular embodiment, the salts are obtained by adding a mixture of diamines, in other words diamines (b) and (c) are added concomitantly.

According to another particular embodiment, the diamines are added sequentially, i.e. diamine (b) is added, and diamine (c) is then added, or vice versa. In this case, the salts obtained may make it possible to lead to copolymers comprising, or even consisting of, copolymers of block type.

The absolute pressure during the polymerization is preferentially between 0.005 MPa and 0.2 MPa. The temperature during the polymerization is preferentially between 50° C. and 250° C.

Preferentially, during the polymerization, a means for keeping the copolyimide salt particles in motion is used so as to prevent aggregation of these particles. A mechanical means may be used to do this, such as a stirrer, rotation of the reactor or agitation by vibration, or fluidization with a carrier gas.

According to a particular embodiment, the polyimide is obtained by a polymerization involving one or more ammonium carboxylate salts obtained from monomers (a), (b) and (c), and in particular a dry salt. For the purposes of the present invention, the term “dry salt” means that the polymerization is not performed in solution or in suspension in a solvent, nor in the melt. In particular, the polymerization does not involve the addition of solvent to the powder(s) placed in the reactor.

The number-average molar mass Mn of the copolyimides may be between 500 g/mol and 50 000 g/mol.

Control of the number-average molar mass may be obtained:
by using chain limiters, i.e. molecules chosen from monoamines, monoanhydrides, monoacids or diacids in α,β positions such that they can form an anhydride function by dehydration reaction; among the chain limiters, mention may be made of phthalic anhydride, 1,2-benzenedicarboxylic acid or ortho-phthalic acid, acetic acid, propionic acid, benzoic acid, stearic acid, benzylamine, 1-aminopentane, 1-aminohexane, 1-aminoheptane, 1-aminooctane, 1-aminononane, 1-aminodecane, 1-aminoundecane and 1-aminododecane, benzylamine, and mixtures thereof,
via a stoichiometric imbalance r=[compound (a)]/([diamine (b)]+[diamine (c)]),
by using branching agents, i.e. molecules with functionality of greater than 3,
by adjusting the synthetic operating conditions such as the residence time, the temperature, the humidity or the pressure,
by a combination of these various means.

In particular, the stoichiometric imbalance r may range from 1.01 to 1.2. That is to say that the imbalance is in particular linked to an excess of monomer (a), and more particularly of tetracarboxylic acid.

According to a particular embodiment, the monomers are, and in particular the salt is:
supplemented with at least one chain limiter and/or
supplemented with an excess of one of the monomers, in particular of monomer (a), or even of carboxylic acid, so as to create a stoichiometric imbalance, i.e. such that r is other than 1.

According to a variant, the chain limiter and/or the stoichiometric excess is added to the salt of step (a) already formed.

According to another variant, the chain limiter and/or the stoichiometric excess of one of the monomers is also in salt form, and in particular it forms a salt with the aliphatic diamine and/or with the tetracarboxylic acid. It may thus be a salt having a stoichiometric imbalance and/or a co-salt or mixed salt of the aliphatic diamines, of tetracarboxylic acid and of chain limiter. Most particularly, the chain limiter and/or the stoichiometric excess is present during the formation of the salt of step (a) and is added at the same time as the species corresponding thereto, for example the limiter of acid type is in a mixture with the tetracarboxylic acid and the limiter of amine type is in a mixture with the aliphatic diamine.

In this second case, the chain limiter allows the formation of salt, and may be chosen especially from the above lists, with the exception of the anhydrides.

The content of chain limiter may range from 0.1% to 10% as a number of moles, especially from 1% to 5% as a number of moles, relative to the total number of moles of monomers, i.e. of monomers (a), (b) and (c) and chain limiter, or even more particularly tetracarboxylic acid, diamine and chain limiter.

When a chain limiter is used, the amounts of amines and of acids may be equilibrated, i.e. the sum of the amine functions is substantially equal to half the sum of acid functions with which they may react. The term “substantially equal” means a maximum difference of 1%.

When a chain limiter is used, the amounts of amines and of acids may be imbalanced, i.e. the sum of the amine functions is substantially different from half the sum of acid functions with which they may react. The term “substantially different” means a difference of at least 1%.

A subject of the invention is thus also a salt of tetracarboxylic acid and of diamines:
in which a chain limiter is also present and/or
which has a stoichiometric imbalance, especially an excess of tetracarboxylic acid,
   and also to the use of such a salt for forming a (co)polyimide and to a process for preparing (co)polyimide using such a salt.

Control of the stoichiometry may be performed at any point in the manufacturing process.

Use may be made of catalysts, added at any point in the process, for instance as a mixture with compound (a), diamine (b) and/or diamine (c), as a mixture with the salt formed either as a solution or by impregnation in the solid state.

It is also possible to perform a polymerization in the melt to obtain polyimides, as described, for example, in patent U.S. Pat. No. 2,710,853. A solvent polymerization may also be performed, especially by following the conventional routes for synthesizing polyimides in solvent, in two steps, for example proceeding via a polyamic acid.

### Compositions

The copolyimide of the invention may be used to make compositions that are generally obtained by mixing the various compounds, fillers and/or additives. The process is performed at more or less high temperature and at more or less high shear force, according to the nature of the various compounds. The compounds can be introduced simultaneously or successively. Use is generally made of an extrusion device in which the material is heated, then melted and subjected to a shear force, and conveyed. According to particular embodiments, it is possible to prepare preblends, optionally in the melt, before preparation of the final composition. It is possible, for example, to prepare a preblend in a resin, for example of the copolyimide, so as to make a masterbatch.

The invention thus also relates to a process for manufacturing a composition by mixing, optionally in the melt, the copolyimide with reinforcing or bulking fillers, and/or impact modifiers and/or additives. The invention also relates to a composition comprising at least the copolyimide, reinforcing or bulking fillers and/or impact modifiers and/or additives.

The composition according to the invention may comprise one or more other polymers.

The composition according to the invention may comprise between 20% and 90% by weight, preferentially between 20% and 70% by weight and more preferentially between 35% and 65% by weight of copolyimide according to the invention, relative to the total weight of the composition.

The composition can additionally comprise reinforcing or bulking fillers. Reinforcing or bulking fillers are fillers conventionally used for the production of thermoplastic compositions, especially based on polyamide. Mention may in particular be made of reinforcing fibrous fillers, such as glass fibers, carbon fibers or organic fibers, non-fibrous fillers such as particulate or lamellar fillers and/or exfoliable or non-exfoliable nanofillers, for instance alumina, carbon black, clays, zirconium phosphate, kaolin, calcium carbonate, copper, diatomaceous earths, graphite, mica, silica, titanium dioxide, zeolites, talc, wollastonite, polymeric fillers, such as, for example, dimethacrylate particles, glass beads or glass powder. Preferably, reinforcing fibers, such as glass fibers, are in particular used.

The composition according to the invention can comprise between 5% and 60% by weight of reinforcing or bulking fillers and preferentially between 10% and 40% by weight, relative to the total weight of the composition.

The composition according to the invention comprising the copolyimide as defined above may comprise at least one impact modifier, i.e. a compound that is capable of modifying the impact strength of a copolyimide composition. These impact modifiers preferentially comprise functional groups which react with the copolyimide. According to the invention, the term “functional groups which react with the copolyimide” means groups that are capable of reacting or of interacting chemically with the residual anhydride, acid or amine functions of the copolyimide, in particular by covalency, ionic or hydrogen bond interaction or van der Waals bonding. Such reactive groups make it possible to ensure good dispersing of the impact modifiers in the copolyimide matrix. Examples that may be mentioned include anhydride, epoxide, ester, amine and carboxylic acid functions and carboxylate or sulfonate derivatives.

The composition according to the invention may also comprise additives normally used for the manufacture of polyimide or polyamide compositions. Thus, mention may be made of lubricants, flame retardants, plasticizers, nucleating agents, anti-UV agents, catalysts, antioxidants, antistatic agents, dyes, mattifying agents, molding aids or other conventional additives.

These fillers, impact modifiers and additives may be added to the copolyimide via suitable usual means that are well known in the field of technical plastics, for instance during salification, after salification, during polymerization, or as a molten mixture.

The copolyimide compositions are generally obtained by blending the various compounds included in the composition without heating or in the melt. The process is performed at more or less high temperature and at more or less high shear force, according to the nature of the various compounds. The compounds can be introduced simultaneously or successively. Use is generally made of an extrusion device in which the material is heated, then melted and subjected to a shear force, and conveyed.

It is possible to blend all the compounds in the molten phase during a single operation, for example during an extrusion operation. It is possible, for example, to blend granules of the polymer materials, to introduce them into the extrusion device in order to melt them and to subject them to more or less high shearing. According to specific embodiments, it is possible to preblend some of the compounds, in the melt or not in the melt, before preparation of the final composition.

### Applications

The copolyimide or the various compositions according to the invention may be used for any forming process for the manufacture of plastic articles. In particular, in the case where good fluidity is desirable, such as melt extrusion, the (co)polyimide may be imbalanced and/or may comprise chain limiters.

The invention thus also relates to a process for manufacturing plastics articles, using the copolyimides of the invention. To this end, mention may be made of various techniques such as the molding process, especially injection molding, extrusion, extrusion blow-molding, or alternatively rotary molding, especially in the field of motor vehicles or of electronics and electricity, for example. The extrusion process may especially be a spinning process or a process for manufacturing films.

The present invention relates, for example, to the manufacture of articles of impregnated fabric type or composite articles containing continuous fibers. These articles may especially be manufactured by placing in contact a fabric and the copolyimide according to the invention in the solid or molten state. Fabrics are textile surfaces obtained by assembling yarns or fibers which are rendered integral by any process, especially such as adhesive bonding, felting, braiding, weaving or knitting. These fabrics are also referred to as fibrous or filamentous networks, for example based on glass fiber, carbon fiber or the like. Their structure may be random, unidirectional (1D) or multidirectional (2D, 2.5D, 3D or other).

Specific language is used in the description so as to facilitate understanding of the principle of the invention. Nevertheless, it should be understood that no limitation on the scope of the invention is envisaged by the use of this specific language. Modifications, improvements and refinements can in particular be envisaged by a person conversant with the technical field concerned on the basis of his own general knowledge.

The term “and/or” includes the meanings and, or and all the other possible combinations of the elements connected to this term.

Other details or advantages of the invention will become more clearly apparent in the light of the examples given below purely by way of indication.

### EXPERIMENTAL SECTION

Measuring standards:

The melting point (Tf) and the crystallization temperature on cooling (Tc) of the copolyimides are determined by differential scanning calorimetry (DSC) by means of a Perkin Elmer Pyris 1 instrument, at a rate of 10° C./min. The Tf and Tc of the copolyimides are determined at the top of the melting and crystallization peaks. The glass transition temperature (Tg) is determined on the same machine at a rate of 40° C./min (when possible, it is determined at 10° C./min and specified in the examples). The measurements are taken after melting the copolyimide formed at T>(Tf of the copolyimide +20° C.).

For the determination of the melting point of the salt, the end temperature of the endotherm measured by heating the salt at 10° C./min is considered.

Thermogravmetric analysis (TGA) is performed on a Perkin-Elmer TGA7 machine on a sample of about 10 mg. The precise conditions of use (temperature, time, heating rate) are defined in the examples.

The Fourier-transform infrared (FTIR) analysis is performed on a Brüker Vector 22 machine (in reflection, ATR Diamant) on the powder of formed copolyimide.

### EXAMPLE 1

### Preparation of Copolyimides PI 10PMA/12PMA of 100/0, 75/25, 50/50, 25/75 and 0/100 mol/mol by Synthesis of Co-Salts

An ethanolic solution of pyromellitic acid is prepared by dissolving 0.00079 mol of pyromellitic acid in 4 mL of absolute ethanol. This solution is added dropwise to a solution heated to 50° C. containing 5 mL of absolute ethanol and 0.00079 mol of a mixture of 1,10-diaminodecane and 1,12-diaminododecane in 100%/0% (Example 1A), 75%/25% (Example 1B), 50%/50% (Example 1C), 25%/75% (Example 1D) and 0%/100% (Example 1E) mole proportions. During the introduction of the pyromellitic acid solution into the diamine mixture, the salt formed precipitates out immediately and is recovered by evaporating off the solvent. The salt is dried overnight under vacuum at 50° C.

The copolyimide formed is prepared by heat treatment above 200° C. of the salt powder and then analyzed by DSC in Table 1 below:

TABLE 1

| | Tf Salt | TfPI | ΔHfPI | TcPI | TgPI* |
|---|---|---|---|---|---|
| PI 10PMA/12PMA | ° C. | ° C. | J/g | ° C. | ° C. |
| 1A (homopolyimide) | 245 | 334 | 47 | 306 | 115 |
| 1B | 242 | 294 | 19 | 274 | 109 |
| 1C | 237 | 269 | 26 | 255 | 104 |
| 1D | 238 | 285 | 30 | 261 | 100 |
| 1E (homopolyimide) | 260 | 303 | 35 | 274 | 96 |

*The Tg is determined at 10° C./min

It is also observed in Table 1 that the copolyimides are semicrystalline and have only one melting point, meaning that they are copolymers that are capable of co-crystallizing. This melting point may be between the Tf values of the two homopolyimides or even lower. It also appears that the heat of fusion is lower than the heat of fusion of the homopolymers but that it remains high irrespective of the molar composition of the diamines. Starting from the copolymerization, it is possible to transform the PI10PMA with a melting point of 334° C. that is difficult to transform via thermoplastic transformation techniques into a semicrystalline polymer with a melting point of less than 300° C. which is much easier to transform.

It will be noted that the FTIR analysis of the copolyimide powder has the characteristic absorption bands of imide functions at 1700 and 1767 cm-1 and the absence of characteristic absorption bands of amide functions is noted.

### EXAMPLE 2

### Preparation of Copolyimides PI 10PMA/13PMA of 100/0, 75/25, 50/50, 25/75 and 0/100 mol/mol by Synthesis of Co-Salts

According to the same procedure as previously, an ethanolic solution of pyromellitic acid is this time added dropwise to a stoichiometric amount of a mixture of 1,10-diaminodecane and 1,13-diaminotridecane dissolved in pure ethanol. The mole ratio chosen for the two C10/C13 diamines is 100%/0% (example 2A), 75%/25% (example 2B), 50%/50% (example 2C), 25%/75% (example 2D) and 0%/100% (example 2E). The salts formed precipitate out immediately and are recovered by evaporating off the solvent, and dried overnight under vacuum at 50° C.

The copolyimide formed is prepared by heat treatment above 200° C. of the salt powder and then analyzed by DSC in Table 2 below:

TABLE 2

| | Tf Salt | TfPI1 | TfPI2 | ΔHfPI1 | ΔHfPI2 | TcPI1 | TcPI2 | TgPI * |
|---|---|---|---|---|---|---|---|---|
| PI 10PMA/13PMA | ° C. | ° C. | ° C. | J/g | J/g | ° C. | ° C. | ° C. |
| 2A (homopolyimide) | 245 | 334 | — | 47 | — | 306 | — | 115 |
| 2B | 254 | 325 | 310 | 15 | 8 | 291 | 291 | N.D. |
| 2C | 234 | 299 | 276 | 5 | 4 | 262 | 205 | N.D. |
| 2D | 238 | 256.7 | 249 | 7 | 7 | 231 | 227 | N.D. |
| 2E (homopolyimide) | 230 | 271 | — | 36 | — | 238 | — | N.D. |

* The Tg is determined at 10° C./min
N.D. = not determined

It is first observed, as for the copolyimides PI 10PMA/12PMA of Example 1, that all the copolyimides PI 10PMA/13PMA are semicrystalline, but it is also observed in Table 2 that they have not just one melting point but two melting points TfPI1 and TfPI2, and associated enthalpies, and two crystallization temperatures TcPI1 and TcPI2. In all cases, they are copolymers, and not mixtures of homopolymers, since:
their melting points are different from the melting points of the homopolymers,
the sum of their associated heats of fusion is less than the sum of the enthalpies of the homopolymers in the proportions under consideration.

### EXAMPLE 3

### Preparation of Copolyimides PI 6PMA/10PMA by Synthesis of Co-Salts or Mixed Salts

The three ethanolic solutions are prepared in the following manner:
Solution 1. 2.807 g of 97.6% pyromellitic acid dissolved in 51.806 g of absolute ethanol. Solution 1 has a concentration of 1.974x10-4 mol/g of pyromellitic acid.
Solution 2. 0.831 g of an aqueous solution of hexamethylenediamine (C6 diamine) at 32.25% by weight dissolved in 16.754 g of ethanol. Solution 2 has a concentration of 1.311×10-4 mol/g of hexamethylenediamine.
Solution 3. 2.202 g of 99% 1,10-diaminodecane (C10 diamine) dissolved in 41.992 g of ethanol. Solution 3 has a concentration of 2.863×10-4 mol/g of 1,10-diaminodecane.

Mixtures of the solutions of diamines 1 and 2 are prepared so as to have mole proportions of C6/C10 diamines equal to 0%/100% (Example 3A), 10%/90% (Example 3B), 15%/85% (Example 3C), 20%/80% (Example 3D) and 30%/70% (Example 3E). These diamine mixtures are then added with stirring to an amount of solution 1 so as to have a stoichiometric amount of diamines (0.0024 mol) and of pyromellitic acid (0.0024 mol). Stirring is maintained for 30 minutes. The salt formed precipitates out and is recovered by evaporating off the solvent, and then dried overnight under vacuum at 45° C.

The copolyimide formed is prepared by heat treatment at 200° C. for 4 hours of the salt powder while flushing with nitrogen, and then analyzed by DSC in Table 3.

TABLE 3

| | TfPI1 | TfPI2 | ΔHfPI1 | ΔHfPI2 | TcPI |
|---|---|---|---|---|---|
| PI 6PMA/10PMA | ° C. | ° C. | J/g | J/g | ° C. |
| 3A (homopolyimide) | 338 | 326 | 27 | 18 | 305 |
| 3B | 329 | 319 | 12 | 22 | 300 |
| 3C | 326 | 316 | 9 | 22 | 295 |
| 3D | 322 | 310 | 5 | 24 | 288 |
| 3E | 325 | 313 | 6 | 16 | 289 |

With this synthetic process, we obtain a double melting peak for PI 10PMA. It is observed in Table 3 that the copolyimides PI 6PMA/10PMA in the zone of molar compositions ranging from 0%/100% to 30%/70% are all semicrystalline. They have two melting points that are different and above all below the melting points of the homopolyimide PI 10PMA (Tf=338° C. and Tf=326° C.), meaning that they are indeed copolymers (insertion of C6 diamine into the PI 10PMA chain) and not mixtures of homopolymers. It is also seen that the sum of the heats of fusion of the two melting peaks of the copolymers is less than the sum of the heats of fusion of the two melting peaks of the homopolyimide PI 10PMA, but that it remains high irrespective of the molar composition of the diamines. Starting from the copolymerization by preparation of PI 6PMA/10PMA co-salts according to this procedure, it is possible to lower the highest melting point of PI 10PMA to about 322° C. (−16° C.).

### EXAMPLE 4

### Preparation of Copolyimides PI 6PMA/10PMA by Sequential Addition of the Monomers

In contrast with Example 3 in which a mixture of C6 and C10 diamines is introduced into a pyromellitic acid solution, a sequential introduction of the diamines into the pyromellitic acid solution is performed here in Example 4:
To begin with, the ethanolic solution of hexamethylenediamine (solution 2) is introduced into the pyromellitic acid solution (solution 1).
The ethanolic solution of 1,10-diaminodecane (solution 3) is then introduced into the mixture of solution 1 and solution 2 thus constituted.
Stirring is maintained for 30 minutes. The salt formed precipitates out and is recovered by evaporating off the solvent, and then dried overnight under vacuum at 45° C.

The introductions are performed so as to have finally 0.0024 mol of pyromellitic acid and 0.0024 mol of diamines. The molar proportions of C6/C10 diamines are respectively equal to 0%/100% (Example 4A), 10%/90% (Example 4B), 15%/85% (Example 4C), 20%/80% (Example 4D) and 30%/70% (Example 4E).

The copolyimide formed is prepared by heat treatment at 200° C. for 4 hours of the salt powder while flushing with nitrogen, and then analyzed by DSC in Table 4 below.

TABLE 4

| | TfPI1 | TfPI2 | ΔHfPI1 | ΔHfPI2 | TcPI |
|---|---|---|---|---|---|
| PI 6PMA/10PMA | ° C. | ° C. | J/g | J/g | ° C. |
| 4A (homopolyimide) | 338 | 326 | 27 | 18 | 306 |
| 4B | 333 | 322 | 19 | 16 | 303 |
| 4C | 334 | 323 | 12 | 22 | 303 |
| 4D | 338 | 326 | 13 | 16 | 303 |
| 4E | 334 | 323 | 17 | 15 | 308 |

It is seen that the melting points and crystallization temperatures of PI 10PMA are virtually unchanged irrespective of the mole proportions of C6 diamine. It is seen, by comparison of the thermal properties of the copolymers of Examples 3 and 4, that the mode of introduction of the monomers and comonomers gives rise to different structures: rather statistical in Example 3 and rather block in Example 4.

1-18. (canceled)

19. A semiaromatic semicrystalline thermoplastic copolyimide obtained by polymerization of at least:
(a) an aromatic compound comprising two anhydride functions and/or carboxylic acid and/or ester derivatives thereof;
(b) a diamine of formula (I) NH2—R—NH2 selected from the group consisting of: 1,4-diaminobutane, 1,5-diaminopentane, 2-methyl-1,5-diaminopentane, hexamethylenediamine, 3-methylhexamethylenediamine, 2,5-dimethylhexamethylenediamine, 2,2,4- and 2,4,4-trimethyl-hexamethylenediamine, 1,7-diaminoheptane, 1,8-diaminooctane; and
(c) a diamine of formula (II) NH2—R′—NH2 in which R′ is a saturated or unsaturated, linear or branched, divalent aliphatic hydrocarbon-based radical, optionally comprising heteroatoms, wherein the two amine functions are separated by a number Y of carbon atoms, Y is between 10 and 20, and the radical R′ comprises not more than 20 carbon atoms;
wherein the copolyimide has at least two melting points Tf, and the melting points are between 50° C. and 330° C., measured by differential scanning calorimetry and heating the copolyimide from 20° C. at a rate of 10° C./minute.

20. The copolyimide as claimed in claim 19, wherein the copolyimide is obtained with addition of chain limiter(s) and/or supplemented with an excess of one of the monomers, so as to create a stoichiometric imbalance.

21. The copolyimide as claimed in claim 19, wherein the copolyimide has two melting points Tf between 50° C. and 330° C., measured by differential scanning calorimetry and heating the copolyimide from 20° C. at a rate of 10° C./minute.

22. The copolyimide as claimed in claim 19, wherein the copolyimide has a glass transition temperature Tg of between −50° C. and +170° C.

23. The copolyimide as claimed in claim 19, wherein the aromatic compound comprising two anhydride functions is selected from the group consisting of: pyromellitic anhydride, 3,3′,4,4′-biphenyltetracarboxylic dianhydride, 2,3,3′,4′-biphenyltetracarboxylic dianhydride, 2,2′,3,3′-biphenyltetracarboxylic dianhydride, 3,3′,4,4′-benzophenonetetracarboxylic dianhydride, 2,2′,3,3′-benzophenonetetracarboxylic dianhydride, 1,2,5,6-naphthalenetetracarboxylic dianhydride, 2,3,6,7-naphthalenetetracarboxylic dianhydride and 2,2′-bis(3,4-dicarboxyphenyl)hexafluoropropanetetracarboxylic dianhydride.

24. The copolyimide as claimed in claim 19, wherein the aromatic compound comprising carboxylic acid functions derived from the two anhydride functions is selected from the group consisting of: pyromellitic acid, 3,3′,4,4′-biphenyltetracarboxylic acid, 2,3,3′,4′-biphenyltetracarboxylic acid, 2,2′,3,3′-biphenyltetracarboxylic acid, 3,3′,4,4′-benzophenonetetracarboxylic acid, 2,2′,3,3′-benzophenonetetracarboxylic acid, 1,2,5,6-naphthalenetetracarboxylic acid, 2,3,6,7-naphthalenetetracarboxylic acid, 2,3,5,6-pyridinetetracarboxylic acid, 3,4,9,10-perylenetetracarboxylic acid, 3,3′,4,4′-tetraphenylsilanetetracarboxylic acid, 2,2′-bis(3,4-bicarboxyphenyl)hexafluoropropanetetracarboxylic acid.

25. The copolyimide as claimed in claim 19, wherein the diamine (b) is selected from the group consisting of: 1,4-diaminobutane, 1,5-diaminopentane, 2-methyl-1,5-diaminopentane, and hexamethylenediamine.

26. The copolyimide as claimed in claim 19, wherein the diamine (c) is selected from the group consisting of: 1,10-diaminodecane, 1,11-diaminoundecane, 1,12-diaminododecane, 1,13-diaminotridecane, 1,14-diaminotetradecane, 1,15-diaminopentadecane, 1,16-diaminohexadecane, 1,17-diaminoheptadecane, 1,18-diaminooctodecane, 1,19-diaminononadecane and 1,20-diaminoeicosane.

27. The copolyimide as claimed in claim 19, wherein the number-average molar mass Mn of the copolyimide is between 500 g/mol and 50,000 g/mol.

28. A process for manufacturing a copolyimide, comprising copolymerizing at least:
(a) an aromatic compound comprising two anhydride functions and/or carboxylic acid and/or ester derivatives thereof;
(b) a diamine of formula (I) NH2—R—NH2 selected from the group consisting of: 1,4-diaminobutane, 1,5-diaminopentane, 2-methyl-1,5-diaminopentane, hexamethylenediamine, 3-methylhexamethylenediamine, 2,5-dimethylhexamethylenediamine, 2,2,4- and 2,4,4-trimethyl-hexamethylenediamine, 1,7-diaminoheptane, 1,8-diaminooctane; and
(c) a diamine of formula (II) NH2—R′—NH2 in which R′ is a saturated and/or unsaturated, linear or branched, divalent aliphatic hydrocarbon-based radical, optionally comprising heteroatoms, the two amine functions being separated by a number Y of carbon atoms, Y being between 10 and 20, the radical R′ comprising not more than 20 carbon atoms;
wherein the copolyimide has at least two melting points Tf, and the melting points are between 50° C. and 330° C., measured by differential scanning calorimetry and heating the copolyimide from 20° C. at a rate of 10° C./minute.

29. A composition comprising at least one copolyimide as claimed in claim 19 and reinforcing or bulking fillers and/or impact modifiers and/or additives.

30. A process for producing a plastics article, comprising forming at least one copolyimide as claimed in claim 19.

31. The process of claim 28, wherein the step of forming comprises injection molding, melt extrusion, extrusion-blow molding, or rotary molding of the polyamide or placing the polyamide, in the solid or molten state, in contact with a fabric.

32. A salt of tetracarboxylic acid and of diamines in which a chain limiter is also present and/or which has a stoichiometric imbalance.

33. A process for preparing a (co)polyimide comprising polymerizing a salt according to claim 32.

34. The process according to claim 28, wherein the diamine of formula (I) is mixed with the diamine of formula (II), and then the mixture comprising the diamine of formula (I) and diamine of formula (II) is added to the aromatic compound.
